(19) United States
(12) Patent Application Publication
(10) Pub. No.: US 2020/0369774 A1
(43) Pub. Date: Nov. 26, 2020

(54) IL-6R ANTIBODY AND ANTIGEN BINDING FRAGMENT THEREOF AND MEDICAL USE

(71) Applicants: Jiangsu Hengrui Medicine Co., Ltd., Lianyungang, Jiangsu (CN); Shanghai Hengrui Pharmaceutical Co., Ltd., Shanghai (CN)

(72) Inventors: Hua Ying, Shanghai (CN); Houcong Jin, Shanghai (CN); Qiyue Hu, Shanghai (CN); Hu Ge, Shanghai (CN); Yifang Wang, Shanghai (CN); Weikang Tao, Shanghai (CN)

(73) Assignees: Jiangsu Hengrui Medicine Co., Ltd., Lianyungang, Jiangsu (CN); Shanghai Hengrui Pharmaceutical Co., Ltd., Shanghai (CN)

(21) Appl. No.: 16/646,673

(22) PCT Filed: Sep. 12, 2018
(86) PCT No.: PCT/CN2018/105180

§ 371 Date: Mar. 12, 2020

(30) Foreign Application Priority Data
Sep. 13, 2017 (CN) 201710821975.4

(51) Int. Cl. C07K 16/28

(52) U.S. Cl. CPC C07K 16/2866; C07K 2317/565; C07K 2317/569; C07K 2317/94; C07K 2317/52; C07K 2317/76; C07K 2317/92; C07K 2317/24

(57) ABSTRACT

Provided in the present invention are an IL-6R antibody and antigen-binding fragment thereof. The IL-6R antibody is an alpaca-derived antibody, chimeric antibody and humanized antibody. Also provided in the present invention is the use of the antibody in the preparation of a drug for treating IL-6-related diseases.

### FIELD OF THE INVENTION

The present disclosure relates to an IL-6R antibody and antigen-binding fragment thereof. Further, the present disclosure also relates to chimeric antibodies and humanized antibodies comprising the CDR regions of the IL-6R antibody. The present disclosure also relates to a pharmaceutical composition comprising the IL-6R antibody and antigen-binding fragment thereof, and its use as a diagnostic and therapeutic agent for diseases associated with IL-6R- or IL-6.

### BACKGROUND OF THE INVENTION

Interleukin-6 (IL-6) is a pleiotropic inflammatory cytokine which regulates growth and differentiation of cells and plays an important role in mediating inflammatory response and immune response. The production of interleukin-6 and its receptor (IL-6R) is associated with the pathogenesis of various diseases such as multiple myeloma, autoimmune diseases and prostate cancer.

IL-6R is the first discovered member of the hematopoietic cytokine receptor superfamily, also known as CD126, including IL-6Ra and IL-6R13, i.e., signal transduction protein gp130 shared by the IL-6 family members. IL-6 binds to IL-6Ra to form an IL-6/IL-6Ra complex, which in turn binds to gp130 to form a high affinity complex (Journal of Immunology, 2006, 25(5): 475-479).

IL-6Ra is mainly expressed on the surface of hepatocytes, neutrophils, macrophages and some lymphocytes; while gp130 is expressed on the surface of all cells (Rheumatology (Oxford, England), 2010, 49(1): 15-24). In addition, there is a soluble form of IL-6R (i.e., sIL-6R). sIL-6R is transported in body fluids, and thus increases the cell type which responds to IL-6. For example, endothelial cells and synovial cells express gp130 but do not express IL-6R. Only in the presence of sIL-6R can these cells respond to IL-6 (Rheumatology (Oxford, England), 2010, 49(1): 15-24). In addition, there is a soluble fragment (sgp130) consisting of the extracellular domain of gp130, which is capable of binding to IL-6 and sIL-6R complex and has an antagonistic effect against the biological activity of IL-6.

Studies have shown that IL-6R is highly expressed in a variety of diseases, such as multiple myeloma, liver cancer and almost all types of myeloid leukemia (Journal of Experimental Hematology 2001: 9 (2) 184-187). As the target of immunotherapy, the first IL-6R monoclonal antibody in the world, Tocilizumab, was approved for marketing in 2009. The second IL-6R monoclonal antibody, Sarilumab, was also first approved for marketing in Canada in January 2017. The IL-6R antigen has been disclosed for a long time. Currently, antibodies against IL-6R have been reported in the patents such as WO1996011020A1, WO2009052454, WO2016089886, WO2005061000, US8753634, US20100215664, U.S. Pat. Nos. 7,582,298, 5,795,965, 8,034,344, WO2016062766 and CN101454345B. Most patents are related to indications and applications of IL-6R antibodies, such as liver cancer, central nervous system (CNS) inflammation, chronic rheumatoid arthritis and vasculitis.

However, there is still a need for improvement of the above-mentioned drugs in the treatment of IL-6-mediated diseases and the like. Therefore, it needs to continue to develop antibodies with high selectivity, good efficacy and low side effects, and provide more and better therapeutic regimen with anti-IL-6R for the treatment of diseases associated with IL-6R.

### SUMMARY OF THE INVENTION

The present disclosure provides a monoclonal antibody or antigen-binding fragment thereof which binds to the amino acid sequence or the three-dimensional structure of IL-6R extracellular region. Furthermore, the present disclosure provides anti-human IL-6R antibodies with high activity and high stability, which compete with said monoclonal antibody or antibody fragment thereof.

Further, the present disclosure provides a method for producing clone of the antibody according to the present disclosure, DNA encoding the antibody, a vector comprising the DNA, and a transformant obtained by transforming the vector. The present disclosure also provides a method of producing the antibody or antibody fragment thereof with the clone or the transformant; and the present disclosure also provides a diagnostic or therapeutic agent comprising the antibody or antibody fragment thereof according to the present disclosure as an active ingredient.

In one aspect, the disclosure provides a monoclonal antibody or antigen-binding fragment thereof. The monoclonal antibody or antigen-binding fragment thereof according to the present disclosure comprises CDR1, CDR2 and CDR3, wherein the sequence of CDR1 is selected from the group consisting of SEQ ID NO: 15 and a variant sequence of SEQ ID NO: 15. In a specific embodiment, the variant sequence of SEQ ID NO: 15 has 1, 2 or 3 amino acid differences from the sequence as set forth in SEQ ID NO: 15. In a specific embodiment, the variant sequence of SEQ ID NO: 15 is obtained via antibody affinity maturation test. The sequence of CDR2 is selected from the group consisting of SEQ ID NO: 16 and a variant sequence of SEQ ID NO: 16. In a specific embodiment, the variant sequence of SEQ ID NO: 16 has 1, 2 or 3 amino acid differences from the sequence as set forth in SEQ ID NO: 16. In a specific embodiment, the variant sequence of SEQ ID NO: 16 is obtained via antibody affinity maturation test. The sequence of CDR3 is selected from the group consisting of SEQ ID NO: 17 and a variant sequence of SEQ ID NO: 17. In a specific embodiment, the variant sequence of SEQ ID NO: 17 has 1, 2 or 3 amino acid differences from the sequence as set forth in SEQ ID NO: 17. In a specific embodiment, the variant sequence of SEQ ID NO: 17 is obtained via antibody affinity maturation test. The monoclonal antibody or antigen-binding fragment thereof recognizes and binds to human IL-6R.

In some embodiments, in the monoclonal antibody or antigen-binding fragment thereof, the CDR1 sequence is set forth in SEQ ID NO: 15; the CDR2 sequence is set forth in SEQ ID NO: 16; and the CDR3 sequence is set forth in SEQ ID NO:17.

In some embodiments, the monoclonal antibody is a recombinant antibody.

In some embodiments, the monoclonal antibody is selected from the group consisting of alpaca-derived antibody, chimeric antibody, humanized antibody and antigen-binding fragment thereof.

In some embodiments, the FR region sequence on the variable region of the humanized antibody is derived from human germline heavy chain or mutated sequence thereof

In some embodiments, the monoclonal antibody comprises VHH of SEQ ID NO: 14 or a variant thereof the variant has 1 to 15 amino acid mutations in the VHH sequence as set forth in SEQ ID NO: 14. More specifically, the variant of VHH has 1 to 15 amino acid mutations in the FR region of VHH as set forth in SEQ ID NO: 14. More specifically, the variant of VHH comprises mutation(s) selected from the group consisting of A14P, E23A, Q44G, V78L, K86R, N96A, A97F, Q116L and combination thereof, in the VHH sequence as set forth in SEQ ID NO: 14.

In some embodiments, the monoclonal antibody or antigen-binding fragment thereof further comprises a human antibody Fc region; preferably comprises a human antibody Fc region as set forth in SEQ ID NO: 19.

In some embodiments, the antigen-binding fragment is selected from the group consisting of a single domain antibody and a peptide comprising the CDR1 to CDR3.

In some embodiments, a monoclonal antibody or antigen-binding fragment thereof according to the present disclosure competes with the monoclonal antibody or antigen-binding fragment thereof as defined above for binding to IL-6R and blocks the binding of IL-6 to IL-6R.

In another aspect, the present disclosure provides a pharmaceutical composition comprising a therapeutically effective amount of the above monoclonal antibody or antigen-binding fragment thereof, and one or more pharmaceutically acceptable carriers, diluents or excipients.

In another aspect, the present disclosure provides a nucleic acid molecule encoding the above monoclonal antibody or antigen-binding fragment thereof.

In some embodiments, the nucleic acid molecule encodes the CDR regions described above. In some embodiments, the nucleic acid molecule comprises polynucleotide having at least 80%, at least 85%, at least 90%, at least 92%, at least 94%, at least 95%, at least 96%, at least 97%, at least 98%, at least 99% or at least 100% homology to SEQ ID NO:13.

In another aspect, the present disclosure provides a recombinant vector comprising the above nucleic acid molecule.

In another aspect, the present disclosure provides a host cell transformed with the above recombinant vector; the host cell is selected from the group consisting of prokaryotic cells and eukaryotic cells, preferably eukaryotic cells, more preferably mammalian cells or yeast cells. The mammalian cells are preferably HEK293E cells, CHO cells or NSO cells. The mammalian cells will not develop into an individual.

In another aspect, the present disclosure provides a method for producing the above monoclonal antibody or antigen-binding fragment thereof, the method comprises the steps of:

cultivating the above host cell;

forming and accumulating the above monoclonal antibody or antigen-binding fragment thereof in the culture; and

recovering the monoclonal antibody or antigen-binding fragment thereof from the culture.

In another aspect, the present disclosure provides a method for detecting or determining human IL-6R, the method comprises the step of detecting IL-6R in a sample by the monoclonal antibody or antigen-binding fragment thereof described above.

In another aspect, the present disclosure provides an agent for detecting or determining human IL-6R, the agent comprises the above monoclonal antibody or antigen-binding fragment thereof.

In another aspect, the present disclosure provides a method for treating a disease associated with human IL-6R, the method comprises administering to a subject the monoclonal antibody or antigen-binding fragment thereof as described above, or administering to a subject a pharmaceutical composition comprising the monoclonal antibody or antigen-binding fragment thereof, or administering to a subject the nucleic acid molecule as described above, for treating a disease associated with human IL-6R.

In another aspect, the present disclosure provides the use of the above monoclonal antibody or antigen-binding fragment thereof for the manufacture of a therapeutic agent for the treatment of a disease associated with IL-6; the present disclosure also provides the use of the above pharmaceutical composition for the manufacture of a therapeutic agent for the treatment of a disease associated with IL-6; the present disclosure provides the use of the above nucleic acid molecule for the manufacture of a therapeutic agent for the treatment of a disease associated with IL-6.

Furthermore, the present disclosure includes an agent for treating a disease associated with IL-6R-positive cells, the agent comprises the monoclonal antibody or antibody fragment thereof according to the present disclosure as an active ingredient.

The antibody, antigen-binding fragment and composition of the present disclosure can be used to prevent and treat a disease and a condition associated with IL-6R, IL-6, and/or associated with IL-6/IL-6R complex (optionally a complex further bound to gp130), and/or associated with signaling pathway and/or biological functions and responses involving IL-6 and/or IL-6/IL-6R complex (optionally a complex further bound to gp130). In particular, the antibody, antigen-binding fragment and composition of the present disclosure can be used to prevent and treat a disease and a condition associated with IL-6R, IL-6, and/or associated with IL-6/IL-6R complex (optionally a complex further bound to gp130), and/or associated with signaling pathway and/or biological functions and responses involving IL-6R, IL-6 and/or IL-6/IL-6R complex (optionally a complex further bound to gp130), wherein the disease and the condition is characterized by excess and/or unwanted signaling mediated by IL-6R or by pathway involving IL-6R. According to the present disclosure, those skilled in the art will apparently understand the examples of the disease and a condition associated with IL-6R, IL-6, and/or associated with IL-6/IL-6R complex, and/or associated with signaling pathway and/or biological functions and responses involving IL-6 and/or IL-6/IL-6R complex. The disease and the condition are also commonly referred to herein as “condition associated with IL-6” or “disease associated with IL-6R”.

In another aspect, the present disclosure provides use of the above monoclonal antibody or antigen-binding fragment thereof, the above pharmaceutical composition, the nucleic acid molecule as described above, or combination thereof, for preparing a medicament for preventing or treating a disease or condition selected from the group consisting of: Sepsis (Starnes et al, 1999) and various cancers, such as multiple myeloma (MM), renal cell carcinoma (RCC), plasma cell leukemia (Klein et al, 1991), lymphoma, B-lymphoid hyperplasia disease (BLPD) and prostate cancer. Non-limiting examples of other diseases caused by excess IL-6 production or signaling include bone resorption (osteoporosis) (Roodman et al, 1992; Jilka et al, 1992), cachexia (Strassman et al, 1992), psoriasis, glomerular mesangial proliferative glomerulonephritis, Kaposi's sarcoma, AIDS-related lymphoma (Emilie et al, 1994); inflammatory diseases and conditions, such as rheumatoid arthritis, systemic onset of juvenile idiopathic arthritis, hypergammaglobulinemia (Grau et al., 1990); regional enteritis, ulcerative colitis, systemic lupus erythematosus (SLE), multiple sclerosis, Castleman's disease, IgMy-globulin disease, cardiac myxoma, asthma (especially allergic asthma), inflammatory anemia, and autoimmune insulin-dependent diabetes (Campbell et al, 1991).

Furthermore, the present disclosure relates to an immunodetection or immunoassay method of IL-6R; reagents for immunodetection or immunoassay of IL-6R; an immunodetection or immunoassay of cells expressing IL-6R; and a diagnostic agent for diagnosing a disease associated with IL-6, which comprises the monoclonal antibody or antibody fragment of the present disclosure as an active ingredient, which specifically recognizes human IL-6R and binds to the amino acid sequence of the extracellular region or the three-dimensional structure thereof.

In the present disclosure, the method for detecting or determining the amount of IL-6R may be any method known in the art. For example, it includes immunodetection or immunoassay.

The immunodetection or immunoassay is a method for detecting or determining the amount of antibody or antigen by using a labeled antigen or antibody. Examples of immunodetection or immunoassay include radioactive substance labeled immunological antibody method (RIA), enzyme immunoassay (EIA or ELISA), fluorescent immunoassay (FIA), luminescent immunoassay, Western Blotting, physicochemical assays, and the like.

The above diseases associated with IL-6 can be diagnosed by detecting or determining the expressed IL-6 with the monoclonal antibody or antibody fragment of the present disclosure.

In order to detect cells expressing the polypeptide, a known immunoassay can be used, preferably immunoprecipitation, fluorescent cell staining, immunohistochemical staining, and the like can be used. Further, a fluorescent antibody staining method with FMAT8100HTS system (Applied Biosystem), and the like can be used.

In the present disclosure, there is not particular limitation for the living sample used for detecting or determining IL-6Rd, as long as it is likely to contain cells expressing IL-6R, for example tissue cells, blood (whole blood, plasma or serum), pancreatic fluid, urine, feces, tissue fluid or culture medium can be used.

The diagnostic agent comprising the monoclonal antibody or antibody fragment thereof of the present disclosure may further comprise an agent for performing antigen-antibody reaction or an agent for detecting the reaction, depending on the desired diagnostic method. The agent for performing antigen-antibody reaction includes such as buffer and salts. The agent for detecting the reaction includes reagents commonly used in immunodetection or immunoassay , for example, such as a labeled secondary antibody recognizing the monoclonal antibody, the antibody fragment thereof or the conjugate thereof, as well as a substrate corresponding to the labels.

### DESCRIPTION OF THE DRAWINGS

FIGS. 1A to 1C display an assay of blocking the binding between IL-6 and IL-6R, by 1764 and humanized antibodies thereof.

FIGS. 2A to 2B display an assay of blocking the binding between gp130 and IL-6/IL-6R, by 1764 and humanized antibodies thereof.

FIG. 3 displays a binding assay of 1764 and U266B1 cell.

FIG. 4 displays an assay of inhibiting IL-6-induced TF-1 proliferation by 1764 antibody.

### DETAILED DESCRIPTION OF THE INVENTION

### 1. TERMINOLOGY

In order to more easily understand the present disclosure, certain technical and scientific terms are specifically defined below. Unless otherwise defined explicitly herein, all other technical and scientific terms used herein have the meaning commonly understood by one of ordinary skilled in the art to which this disclosure belongs.

The three-letter code and the one-letter code for amino acids used in the present disclosure are as described in J. biol. chem, 243, p3558 (1968).

A conventional immunoglobulin is a tetramer composed of two heavy chains and two light chains, with a combined molecular weight of about 150 kDa. A significant proportion of serum antibodies in Camelidae members are homodimeric IgGs, with a molecular weight of approximately 80 kD (Hamers-Casterman et al. 1993 Nature, 363, 446-448). These heavy chain immunoglobulins (Igs) comprise three domains, the variable regions of which are referred to as VHHs (variable domains of heavy chain of heavy-chain antibody). Recombinant VHH (approximately 12 to 14 kD) constitutes the entire antigen binding domain and exhibits a broad antigen binding profile. Expanding their hypervariable regions exhibit unique property, for example three to four hydrophobic framework residues (which interact with conventional antibody VL) are replaced by more hydrophilic amino acids. In order to stabilize the enlarged CDRs, in addition to conventional disulfide bonds, VHH may have additional disulfide bond(s) between CDR1 and CDR3 in the case of dromedary, and between CDR2 and CDR3 in the case of llama (Harmsen and De Haard 2007 Appl Microbiol Biotechnol., 77, 13-22; Muyldermans 2001 J Biotechnol., 74, 277-302). The enlarged CDR3 loop can adopt convex conformation, while the conventional paratope is confined to a concave or planar structure (Muyldermans 2001 J Biotechnol., 74, 277-302). These features allow VHH to recognize unique epitopes which are less immunogenic when compared with conventional antibodies (Lafaye et al. 2009 Mol Immuno., 46, 695-704; Wernery 2001 J Vet Med B Infect Dis Vet Public Health., 48, 561-568). Although VHH is defined as a monovalent antibody, any affinity effect is excluded by default, and the biological activity is measured as in vitro IC50, which is similar to that of a conventional bivalent antibody molecule (Thys et al. 2010 Antiviral Res., 87: 257-264).

The term “monoclonal antibody” refers to an antibody obtained from a population of substantially homologous antibodies, i.e., each antibody comprised in the population is identical (except for possible natural mutations that may be present in minor amount). A monoclonal antibody is highly specific for a single antigenic site. Furthermore, unlike conventional polyclonal antibody preparations, which typically include different antibodies directed against different determinants (epitopes) on the antigen, each monoclonal antibody is directed only to a single determinant or epitope on the antigen.

In certain embodiments, the disclosure may be directed to a chimeric camelidae/human antibody, particularly a chimeric antibody in which the VH and/or VL domain is entirely camelidae sequence (e.g., llama or Alpaca), while the rest of the antibody completely consists of human sequence. In some preferred embodiments of the present disclosure, it further relates to “humanized” or “germlined” camelidae antibody and a chimeric camelidae/human antibody, in which the VH and/or VL domain contains one or more amino acid substitutions in the framework region, relative to the camelidae VH and/or VL domain obtained by active immunization. The percent sequence identity relative to human germline VH or VL domain is increased by such “humanization” process, i.e., replacing the mismatched amino acid residues located in the original camelidae VH or VL domain with the corresponding residues located in the human germline VH or VL domain.

The present disclosure includes natural, recombinant VHH or VH.

“Recombination” involves production of the VHH or VH with genetic engineering methods (cloning, amplification).

The VHH according to the present disclosure may be in the form of a monomer or a homopolymer, such as a homodimer or a homotrimer.

About 110 amino acid sequences adjacent to the N-terminus of the antibody heavy and light chains are highly variable, known as variable region (Fv region); the rest of amino acid sequences close to the C-terminus are relatively conservative, known as constant region. The variable region includes three hypervariable regions (HVRs) and four relatively conserved framework regions (FRs). The three hypervariable regions which determine the specificity of the antibody are also known as the complementarity determining regions (CDRs). Each light chain variable region (LCVR) and each heavy chain variable region (HCVR) consists of three CDR regions and four FR regions, with sequential order from the amino terminus to carboxyl terminus in the following order: FR1, CDR1, FR2, CDR2, FR3, CDR3, and FR4. The three CDR regions of the light chain refer to LCDR1, LCDR2, and LCDR3, and the three CDR regions of the heavy chain refer to HCDR1, HCDR2, and HCDR3. The number and position of CDR amino acid residues in the LCVR and HCVR regions of the antibody or antigen binding fragments herein comply with known Kabat numbering criteria (LCDR1-3, HCDR1-3).

The antibody of the present disclosure includes alpaca-derived antibodies, chimeric antibodies, humanized antibodies, preferably humanized antibodies.

The term “alpaca-derived antibody” is an alpaca-derived monoclonal antibody against human IL-6R and may be prepared according to the knowledge and skill in the art. For preparation of such antibody, the subject is injected with an IL-6R antigen, and then a hybridoma expressing the antibody having the desired sequence or functional property is isolated, or an immunological library is established and the antibody having the desired function is isolated by phage display technology. In a specific embodiment of the present disclosure, the alpaca-derived IL-6R antibody or antigen-binding fragment thereof may further comprise alpaca heavy chain Fc region.

The term “chimeric antibody” is an antibody which is formed by fusing the variable region of an alpaca-derived antibody with the constant region (or Fc region) of a human antibody, and the chimeric antibody has alleviated immune response induced by the alpaca-derived antibody. To construct a chimeric antibody, first, a hybridoma or an antibody library that secretes a specific alpaca-derived monoclonal antibody is constructed, and then alpaca-derived variable region gene is ligated with the human constant region gene (or Fc region) to form a chimeric gene which is subsequently inserted into an expression vector, and finally a chimeric antibody molecule is expressed in the eukaryotic or prokaryotic system. In a preferred embodiment of the present disclosure, the heavy chain of the IL-6R chimeric antibody further comprises the heavy chain constant region (or Fc region) of human IgG1, IgG2, IgG3, or IgG4, or variant thereof, preferably comprises the heavy chain constant region (or Fc region) of human IgG1, IgG2, or IgG4, or a variant of IgG1, IgG2, or IgG4 heavy chain constant region (or Fc region) comprising amino acid mutations (such as YTE mutation or back-mutation)

The term “humanized antibody” refers to an antibody generated by grafting alpaca CDR sequences into a variable region framework of human antibody, namely an antibody produced from different types of human germline antibody framework sequences. A humanized antibody overcomes disadvantage of the heterologous reaction induced by the chimeric antibody, which carries a large amount of alpaca protein components. Such framework sequences can be obtained from a public DNA database covering germline antibody gene sequences or published references. For example, germline DNA sequences of human heavy and light chain variable region genes can be found in “VBase” human germline sequence database (available on web www.mrccpe.com.ac.uk/vbase), as well as can be found in Kabat, E A, et al, 1991 Sequences of Proteins of Immunological Interest, 5th Ed. To avoid a decrease in activity caused by the decreased immunogenicity, the framework sequences in the variable region of human antibody are subjected to minimal reverse mutations or back mutations to maintain the activity. The humanized antibody of the present disclosure also comprises humanized antibody on which CDR affinity maturation is performed by phage display. To avoid a decrease in activity caused by the decreased immunogenicity, on the basis of the sequences of 1764, gradual mutation toward human sequence is performed, that is, gradually replace the alpaca-derived residues located on FR region of 1764 with human-derived residues to maintain the activity.

The term “VHH” relates to variable antigen binding domain derived from a heavy chain antibody of Camelidae (camel, dromedary, llama, alpaca, etc.) (see Nguyen et al. 2000 EMBO J., 19, 921-930; Muyldermans 2001 J Biotechnol., 74, 277-302 and review Vanlandschoot et al. 2011 Antiviral Res. 92, 389-407).

Generally, a Nanobody can be defined as amino acid sequences having the following (general) structure:

FR1-CDR1-FR2-CDR2-FR3-CDR3-FR4

Wherein FR1-FR4 refer to framework regions 1-4, respectively, and wherein CDR1-CDR3 refer to complementarity determining regions 1-3, respectively.

The term “antibody frame region” or “antibody framework region” as used herein, refers to a portion of the variable domain (VH) which serves as a scaffold for the antigen binding loop (CDR) of the variable domain. Essentially, it refers to a variable domain without CDR.

The term “antigen-binding fragment” or “functional fragment” of an antibody refers to one or more fragments of an antibody that retain the ability to specifically bind to an antigen (e.g., IL-6R). It has been shown that antigen binding function of an antibody can be achieved by fragments of the full antibody. Examples of the binding fragment comprised in the term “antigen-binding fragment” of an antibody include:

(i) Fab fragment, a monovalent fragment consisting of VL, VH, CL and CH1 domain;

(ii) F(ab')2 fragment, a bivalent fragment comprising two Fab fragments linked by a disulfide bridge located on the hinge region;

(iii) Fd fragment consisting of VH and CH1 domain;

(iv) Fv fragment consisting of VH and VL domain of a single arm of the antibody;

(v) a single domain or dAb fragment (Ward et al, (1989) Nature 341:544-546), which consists of VI-1H domain;

(vi) an isolated complementarity determining region (CDR); or

(vii) a combination of two or more separate CDRs, optionally linked by a synthetic linker;

(viii) a fusion protein formed by VHH and Fc;

(ix) a fusion protein formed by VHH and antibody heavy chain constant region.

In addition, “antigen-binding fragment” also includes other antigen-binding forms comprising the three CDRs in VHH domain which is capable of binding to IL-6R.

The disclosure further encompasses such an antigen binding polypeptide, in which hypervariable loops or CDRs of the VHH domain are derived from camelidae, while at least one of the hypervariable loops or CDRs derived from camelidae is engineered to contain one or more amino acid substitutions, additions or deletions, relative to the coding sequence derived from camelidae. These changes include the “humanization” of hypervariable loops/CDRs. The camelidae-derived HV/CDRs engineered in this manner can still exhibit amino acid sequences “substantially identical” to those of the HV/CDRs encoded by camelidae. In this case, “substantially identical” may allow no more than one, or no more than two, or no more than three amino acid sequence mismatches when compared to the HV/CDRs encoded by camelidae.

The antibodies of the present disclosure may be of any isotype. The types of antibodies for human therapeutic use are typically of IgA, IgD, IgE, IgG, IgM type. Usually the antibodies are IgG type antibodies, in such case, they may belong to any one of the four subtypes, i.e., IgGl, IgG2a and IgG2b, IgG3 or IgG4. In these subtypes, one or more amino acid substitutions, insertions or deletions are allowed in the Fc portion, or other structural modifications are allowed to, for example, increase or decrease Fc-dependent function.

The diabody of the present disclosure can be produced by the steps of: obtaining cDNA encoding the VHH of the monoclonal antibody of the present disclosure which specifically recognizes human IL-6R and binds to the extracellular region (amino acid sequence or three-dimensional structure thereof), constructing a DNA encoding the VHH, such that the peptide linker is 8 or less amino acid residues in length, inserting the DNA into a prokaryotic expression vector or a eukaryotic expression vector, and then introducing the expression vector into a prokaryote or eukaryote to express the diabody.

A CDR-containing peptide is constructed by one or more regions of CDRs comprising VHH. Peptides comprising several CDRs can be joined directly or via a suitable peptide linker.

The CDR-containing peptide of the present disclosure can be produced by the steps of: constructing a DNA encoding the CDR of VHH of the monoclonal antibody of the present disclosure which specifically recognizes human IL-6R and binds to the extracellular region (amino acid sequence or three-dimensional structure thereof), inserting the DNA into a prokaryotic expression vector or a eukaryotic expression vector, and then introducing the expression vector into a prokaryote or eukaryote to express the peptide. The CDR-containing peptide can also be produced by a chemical synthesis method such as Fmoc method or tBoc method.

The term “epitope” or “antigenic determinant” refers to a site on an antigen to which an immunoglobulin or antibody specifically binds (e.g., a specific site on the IL-6R molecule). Epitopes typically include at least 3, 4, 5, 6, 7, 8, 9, 10, 11, 12, 13, 14 or 15 consecutive or non-consecutive amino acids in a unique tertiary conformation. See, for example, Epitope Mapping Protocols in Methods in Molecular Biology, Vol. 66, G. E. Morris ed. (1996).

The term “specific binding”, “selective binding”, “selectively binds to” and “specifically binds to” refers to the binding of an antibody to a predetermined epitope on an antigen. Typically, the antibody binds with an affinity (K_{D}) of less than about 10⁻⁷ M, for example, less than about 10⁻⁸ M, 10⁻⁹ M, or 10⁻¹⁰ M or even less.

The term “K_{D}” or “Kd” refers to the dissociation equilibrium constant for particular antibody-antigen interaction. Typically, the antibody of the present disclosure binds to IL-R6 with a dissociation equilibrium constant (KD) of less than about 10⁻⁷ M, such as less than about 10⁻⁸ M, 10⁻⁹ M or 10⁻¹⁰ M or even less, for example, as determined using surface plasmon resonance (SPR) techniques in a BIACORE instrument.

When the term “competition” is used in the context of antigen binding proteins (e.g., neutralizing antigen binding proteins or neutralizing antibodies) that compete for the same epitope, it means that competition occurs between the antigen binding proteins, which is determined by the following assays: the antigen binding protein to be tested (e.g., an antibody or immunologically functional fragment thereof) prevents or inhibits (e.g., reduces) the specific binding between a reference antigen binding protein (e.g., a ligand or reference antibody) and a common antigen (e.g., an IL-6R antigen or fragment thereof). Numerous types of competitive binding assays are available to determine whether an antigen binding protein competes with another. These assays are, for example, solid phase direct or indirect radioimmunoassay (RIA), solid phase direct or indirect enzyme immunoassay (EIA), Sandwich competition assay (see, e.g., Stahli et al, 1983, Methods in Enzymology 9: 242-253); solid phase direct biotin-avidin EIA (see, e.g., Kirkland et al, 1986, J. Immunol. 137: 3614-3619), solid phase direct labeling assay, solid phase direct labeling sandwich assay (see, e.g., Harlow and Lane, 1988, Antibodies, A Laboratory Manual, Cold Spring Harbor Press); solid phase direct labeling RIA with I⁻¹²⁵ label (see, e.g., Morel et al, 1988, Molec. Immunol. 25: 7-15); solid phase direct biotin-avidin EIA (see, e.g., Cheung, et al, 1990, Virology 176: 546-552); and direct labeling RIA (Moldenhauer et al, 1990, Scand. J. Immunol. 32: 77-82). Typically, the assay involves the use of a purified antigen (either on a solid surface or on a cell surface) capable of binding to both an unlabeled antigen binding protein to be tested and a labeled reference antigen binding protein. Competitive inhibition is determined by measuring the amount of label bound to the solid surface or to the cell in the presence of the antigen binding protein to be tested. Usually, the antigen binding protein to be tested is present in excess. Antigen binding proteins identified by competitive assay (competing with the antigen binding protein) includes: antigen binding proteins that bind to the same epitope as the reference antigen binding protein; and antigen binding proteins that bind to an epitope that is sufficiently close to the epitope to which the reference antigen binding protein binds, where the two epitopes spatially interfere with each other to hinder the binding. Additional details regarding methods for determining competitive binding are provided in the Examples herein. Typically, when a competing antigen binding protein is present in excess, it will inhibit (e.g., reduce) at least 40-45%, 45-50%, 50-55%, 55-60%, 60-65%, 65-70%, 70-75% or 75% or even more of the specific binding between the reference antigen binding protein and the common antigen. In some cases, binding is inhibited by at least 80-85%, 85-90%, 90-95%, 95-97%, or 97% or even more.

The term “nucleic acid molecule,” as used herein refers to DNA molecules and RNA molecules. A nucleic acid molecule may be single-stranded or double-stranded, but preferably is double-stranded DNA. A nucleic acid is deemed as “operably linked” when it is placed into a functional relationship with another nucleic acid sequence. For instance, a promoter or enhancer is operably linked to the coding sequence if it affects the transcription of the sequence.

The term “vector” refers to a nucleic acid molecule capable of transporting another nucleic acid with which it has been linked. In one embodiment, the vector is a “plasmid,” which refers to a circular double stranded DNA loop into which additional DNA segments may be ligated. In another embodiment, the vector is a viral vector, wherein additional DNA segments may be ligated into the viral genome. The vectors disclosed herein are capable of self-replicating in the host cell into which they have been introduced (e.g., bacterial vectors having bacterial replication origin and episomal mammalian vectors), or may be integrated into the genome of a host cell upon introduction into the host cell, and thereby are replicated along with the host genome (e.g., non-episomal mammalian vectors).

Methods for producing and purifying antibodies and antigen-binding fragments are well known in the art and can be found, for example, in Antibodies, A Laboratory Manual, Cold Spring Harbor Laboratory Press, chapters 5-8 and 15. For example, mice can be immunized with human IL-6R or fragments thereof, and the resulting antibodies can then be renatured, purified, and sequenced for amino acid sequences by using conventional methods well known in the art. Antigen-binding fragments can also be prepared by conventional methods. The antibodies or antigen binding fragments of the present disclosure are engineered to introduce one or more human framework regions into CDRs derived from a non-human antibody. Human FR germline sequences can be obtained by aligning human antibody variable germline gene database and MOE software, and are available from ImMunoGeneTics (IMGT) website http://imgt.cines.fr, or from The Immunoglobulin Facts Book, 2001, ISBN 012441351.

The term “host cell” refers to a cell into which the expression vector has been introduced. Host cells may include microbial (e.g. bacterial), plant or animal cells. Bacteria that are susceptible to be transformed include members of enterobacteriaceae, such as Escherichia coli or Salmonella strains; Bacillaceae such as Bacillus subtilis; Pneumococcus; Streptococcus and Haemophilus influenzae. Suitable microorganisms include Saccharomyces cerevisiae and Pichia pastoris. Suitable animal host cell lines include, but are not limited to CHO (Chinese hamster ovary cell line), HEK cells (as non-limiting examples, HEK293E cells) and NSO cells, etc.

The engineered antibodies or antigen binding fragments of the present disclosure may be prepared and purified using known methods. For example, cDNA sequence encoding a heavy chain and a light chain may be cloned and recombined into a GS expression vector. The recombinant immunoglobulin expression vector may then be stably transfected into CHO cells. As a more recommended method well known in the art, mammalian expression systems will result in glycosylation, typically at highly conserved N-terminal sites in the Fc region. Stable clones may be verified for expression of antibody specifically binding to human IL-6R. Positive clones may be expanded in serum-free culture medium in bioreactors for antibody production. Culture medium, into which an antibody has been secreted, may be purified by conventional techniques. For example, Protein A or G Sepharose FF column that has been equilibrated with adjusting buffer can be used for purification. The column is washed to remove nonspecific binding components, and then the bound antibody is eluted by pH gradient and antibody fractions are detected, by SDS-PAGE, and then collected. The antibodies may be filtered and concentrated using common techniques. Soluble aggregates and polymers may be effectively removed by common techniques, such as size exclusion or ion exchange. The resulting product is then immediately frozen, for example at −70° C., or may be lyophilized.

“Administration”, “administering” or “treatment,” as it applies to an animal, human, subject, cell, tissue, organ, or biological fluid, refers to contacting an exogenous pharmaceutical, therapeutic, diagnostic agent, or composition with the animal, human, subject, cell, tissue, organ, or biological fluid. “Administration”, “administering” or “treatment” can refer, e.g., to therapeutic, pharmacokinetic, diagnostic, research and experimental methods. Treatment of a cell encompasses contacting a reagent with the cell, as well as contacting a reagent with a fluid, where the fluid is in contact with the cell. “Administration”, “administering” or “treatment” also means in vitro and ex vivo treatments, e.g., of a cell, with a reagent, diagnostic, binding composition, or with another cell. “Treatment,” as it applies to human, veterinary, or research subject, refers to therapeutic treatment, prophylactic or preventative measures, research and diagnostic applications.

“Treat” means to administration of a therapeutic agent, such as a composition containing any of antibodies or fragment thereof of the present disclosure, internally or externally to a patient having one or more disease symptoms for which the agent has known therapeutic activity. Typically, the agent is administered in an amount effective to alleviate one or more disease symptoms in the patient or population to be treated, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in the patient. Whether a disease symptom has been alleviated can be assessed by any clinical measurement typically used by physicians or other skilled healthcare providers to assess the severity or progression status of that symptom. While an embodiment of the present disclosure (e.g., a treatment method or manufacture article) may not be effective in alleviating the target disease symptom(s) in every patient, it should alleviate the target disease symptom(s) in a statistically significant number of patients as determined by any statistical test known in the art such as the Student's t-test, the chi-square test, the U-test according to Mann and Whitney, the Kruskal-Wallis test (H-test), Jonckheere-Terpstra-test and the Wilcoxon-test.

“Conservative modification” or “conservative substitution or replacement” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitution in non-essential regions of a polypeptide does not substantially alter biological activity (see, e.g., Watson et al. (1987) Molecular Biology of the Gene, The Benjamin/Cummings Pub. Co., p. 224 (4th Ed.)). In addition, substitutions with structurally or functionally similar amino acids are less likely to disrupt biological activity.

“Effective amount” encompasses an amount sufficient to ameliorate or prevent a symptom or sign of the medical condition. Effective amount also means an amount sufficient to allow or facilitate diagnosis. An effective amount for a particular patient or veterinary subject may vary depending on factors such as the condition being treated, the overall health condition of the patient, the route and dose of administration and the severity of side effects. An effective amount can be the maximal dose or dosing protocol that avoids significant side effects or toxic effects.

“Exogenous” refers to substances that are produced outside an organism, cell, or human body, depending on the context. “Endogenous” refers to substances that are produced within a cell, organism, or human body, depending on the context.

“Homology” refers to sequence similarity between two polynucleotide sequences or between two polypeptide sequences. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions to be compared, and then multiplied by 100. For example, when the sequences are optimally aligned, if 6 out of 10 positions in two sequences are matched or homologous then the two sequences have 60% homology; if 95 out of 100 positions in two sequences are matched or homologous, then the two sequences have 95% homology. Generally, the comparison is performed when two sequences are aligned to give maximum percent homology.

As used herein, the expressions “cell,” “cell line,” and “cell culture” are used interchangeably and all such designations include progeny thereof Thus, the terms “transformants” and “transformed cells” include the primary subject cells and cultures derived therefrom, regardless of the number of transfers or passages. It should be also understood that all progeny may not be precisely identical in DNA content, due to deliberate or inadvertent mutations. Mutant progeny thus screened that have the same function or biological activity as that of originally transformed cells are included. Where distinct designations are intended, it will be clearly understood from the context.

As used herein, “polymerase chain reaction” or “PCR” refers to a procedure or technique in which minute amount of a specific portion of nucleic acid, RNA and/or DNA, is amplified as described in, e.g., U.S. Pat. No. 4,683,195. Generally, sequence information at the ends of the region of interest or beyond the region of interest is needed, such that oligonucleotide primers can be designed; these primers will be identical or similar in sequence to opposite strands of the template to be amplified. The 5′ terminal nucleotides of the two primers are in consistence with the ends of the material to be amplified. PCR can be used to amplify specific RNA sequences, specific DNA sequences from total genomic DNA, and cDNA transcribed from total cellular RNA, bacteriophage or plasmid sequences, etc. See generally Mlis et al. (1987) Cold Spring Harbor Symp. Ouant. Biol. 51:263; Erlich, ed., (1989) PCR TECHNOLOGY (Stockton Press, N.Y.). The PCR test used in the present disclosure is considered to be one, but not the only, example of polymerase reaction method for amplifying a nucleic acid test sample. The method comprises the use of a known nucleic acid as primers and a nucleic acid polymerase to amplify or generate a specific portion of nucleic acid.

“Optional” or “optionally” means that the event or situation that follows may but does not necessarily occur, and the description includes the instances in which the event or situation does or does not occur. For example, “optionally contains 1-3 antibody heavy chain variable regions” means the antibody heavy chain variable region with specific sequence can be, but need not be, present.

“Pharmaceutical composition” refers to a mixture containing one or more compounds according to the present disclosure or a physiologically/pharmaceutically acceptable salt or produg thereof and other chemical components, such as physiologically/pharmaceutically acceptable carriers and excipients. The pharmaceutical composition aims at promoting the administration to an organism, facilitating the absorption of the active ingredient and thereby exerting a biological effect.

### 2. EXAMPLES AND TEST EXAMPLES

The following examples are provided to further describe the present disclosure, but are not intended to limit the scope of the disclosure.

Experimental methods for which the specific conditions are not indicated in the examples or test examples of the present disclosure are generally carried out according to conventional conditions or according to the conditions recommended by the manufacturer. See Sambrook et al., Molecular Cloning, Laboratory Manual, Cold Spring Harbor Laboratory; Contemporary Molecular Biology Methods, Ausubel et al., Greene Publishing Association, Wiley Interscience, NY. Reagents for which the sources are not specifically indicated are commercially available reagents.

### EXAMPLES

### Example 1: Preparation of Reagents such as Antigens, Related Proteins and Antibodies

Sequences encoding human interleukin-6 receptor with Fc, His and BP15 tags (used for intracellular site-directed biotinylation), respectively (hIL-6R.Fc, hIL-6R.His, hIL-6R.BP15), sequence encoding cynomolgus IL-6R with Flag.His tag (cIL-6R.FH), sequence encoding human IL-6 with Fc or His tag (IL-6.Fc, IL-6.his), and sequence encoding gp130 with Fc-tag were constructed into expression vector(s) by molecular cloning method, for transient expression in mammalian cell(s).

The amino acid sequences of all antigens and related proteins are as follows:

hIL-6R.His
(SEQ ID NO: 1)
LAPRRCPAQEVARGVLTSLPODSVTUTCPGVEPEDNATVHWVLRKPAAGS
HPSRWAGMGRRLLLRSVQLHDSGNYSCYRAGRPAGTVHLLVDVPPEEPQL
SCFRKSPLSNVVCEWGPRSTPSLTTKAVLLVRKFQNSPAEDFQEPCOYSQ
LSQKFSCQLAVPEGDSSFYIVSMCVASSVGSKESKTQTPQGCGILQPDPP
ANITVTAVARNPRWLSVTWQDPHSWNSSFYRLRFELRYRAERSKTFITWM
VKDLQHHCVIHDAWSGLRHVVQLRAQEEFGQGEWSEWSPEAMGTPWTESR
SPPAENEVSTPMQALTTNKDDDNILFRDSANATSLPVQDSSSVPLPHHHH
HH
hIL-6R.BP15
(SEQ ID NO: 2)
LAPRRCPAQEVARGVLTSLPGDSVTLTCPGVEPEDNATVHWVLRKPAAGS
HPSRWAGMGRRLLLRSVQLHDSGNYSCYRAGRPAGTVHLLVDVPPEEPQL
SCFRKSPLSNVVCEWGPRSTPSLTTKAVLLARKFQNSPAEDFQEPCQYSQ
ESQKFSCQLAVPEGDSSFYIVSMCVASSVGSKFSKTQTFQGCGILQPDPP
ANITVTAVARNPRWLSVTWQDPHSWNSSFYRLRFELRYRAERSKTFTTWM
VKDLQHHCVIHDAWSGLRHVVQLRAQEEPGQGEWSEWSPEAMGTPWTESR
SPPAENEVSTPMQALTTNKDDDNILFRDSANATSLPVQDSSSVPLPGSSD
YKDDDDKHHHHHHGLNDIFEAQKIEWHE
hIL-6R.Fc
(SEQ ID NO: 3)
LAPRRCRAQEVARGVLTSLPGDSVTLTCPGVEPEDNATVHWVLRKPAAGS
HPSRWAGMGRRLLLRSVQLHDSGNYSCYRAGRPAGTVHLLVDVPPEEPQL
SCFRKSPLSNVVCEWGPRSTPSLTTKAVLLVRKFQNSPAEDFQEPCQYSQ
ESQKFSCLAVPEGDSSEYIVSMCVASSVGSKFSKTQTFQGCGILQPDPPA
NITVTAVARNPRWLSVTWQDPHSWNSSFYRLRFELRYRAERSKTFTTWMV
KDLQHHCVIHDAWSGLRHVVQLRAQEEFGQGEWSEWSPEAMGTPWTESRS
PPAENEVSSTPMQALTTNKDDDNILFRDSANATSLPVQDSSSVPLPEPPK
SSDKTHTCPPCPAPELLGGPSVFLFPPKPKDTLMISRTPEVTCVVVDVSH
EDPENKFNWYVDGVEVHNAKTKPREEQYNSTYRVVSVLTVLHQDWLNGKE
YKCKVSNKALPAPIEKTISKAKGQPREPQVYTLFPSRDELTKNQVSLTCL
VKOFYPSDIAVEWESNGQPENNYKTTPPVLDSDGSFFLYSKLTVDKSRWQ
QGNVPSCSVMHEALHNHYTQKSLSLSPGK
cIL-6R.FH
(cIL-6R.FlagHis SEQ ID NO: 4)
LAPGGCPAQEVARGVLTSLPGDSVTLTCPGGEPEDNATVHWVLRKPAVGS
HLSRWAGVGRRLLLRSVQLRDSGNYSCYRAGRPAGTVHLLVDVPPEEPQL
SCFRKSPLSNVVCEWGPRSTPSPTTKAVLLVRKFQNSPAEDFQEPCQYSQ
ESQKFSCQLAVPEGDSSFYIVSMCVASSVGSKLSKTQTFQGCGILQPDPP
ANITVTAVARNPRWLSVTWQDPHSWNSSFYRLRFELRYRAERSKTFTTWM
VKDLQHHCVIHDAWSGLRHVVQLRAQEEFGQGEWSEWSPEAMGTPWTESR
SPPAENEVSTPTQAPTTNKDDDNILSRDSANATSLPVQDSSSVPLPGSSD
YKDDDDKHHHHHH
IL-6.Fc
(SEQ ID NO: 5)
VPPGEDSKDVAAPHRQPLTSSERIDKQIRYILDGISALRKETCNKSNMCE
SSKEALAENNLNIPKMAEKDGCFQSGFNEETCLVKITTGLLEFEVYLEYL
QNRFESSEEQARAVQMSTKVLIQFLQKKAKNLDAITTFDPTTNASLLTKL
QAQNQWLQDMTTHLILRSFKEFLQSSLRALRQMEPKSSDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK
IL-6.his
(SEQ ID NO: 6)
VPPGEDSKDVAAPHRQPLTSSERIDKQIRYILDGISALRKETCNKSNMCE
SSKEALAENNLNLPKMAEKDGCFQSGFNEETCLVKIITGLLEFEVYLEYL
QNRFESSEEQARAVQMSTKVLIQFLQKKAKNLDAITTPDPTTNASLLTKL
QAQNQWLQDMTTHLILRSFKEFLQSSLRALRQMHHHHHHH
gp130.Fc
(SEQ ID NO: 7)
ELLDPCGYISPESPVVQLHSNFTAVCVLKEKCMDYFHVNANYIVWKTNHF
TIPKEVYTTINRTASSVTFIDIASLNIQLTCNILTFGQLEQNVYGITIIS
GLPPEKPKNLSCIVNEGKKMRCEWDGGRETHLETNFTLKSEWATHKFADC
KAKRDTPTSCTVDYSTVYFYNIEVWVEAENALGKVTSDHINFDPVYKVKP
NPPHNLSVINSEELSSILKLTWTNPSIKSSVIILKYNIQYRTKDASTWSQ
IPPEDTASTRSSFTVQDLKPFTEYVFRIRCMKEDGKGYWSDWSEEASGIT
YEDRPSKAPSFWYKIDPSHTQGYRTVQLVWKTLPPFEANGKILDYEVTLT
RWKSHLQNYTVNATKLTVNLTNDRYLATLTVRNLNGKSDAAVLTIPACDD
FQATHPVMDLKAFPKDNMLWVEWTTPRESVKKYILEWCVLSDKAPCITDW
QQEDGTVHRTYLRGNLAESKCYLITVTPVYADGPGSPESIKAYLKQAPPS
KGPTVRTKKVGKNEAVLEWDQLPVDVQNGFIRNYTIFYRTIIGNETAVNV
DSSHTEYTLSSLTSDTLYMVRMAAYTDEGGKDGPEFTFTTPKFAQGEIEF
PKSSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK

The positive antibodies are: anti-IL-6R humanized mAb tocilizumab available from Roche/Chugai (the sequence is obtained from WO1996011020A1); a fusion protein formed by anti-IL-6R single domain antibody 20A11 and Fc, Ablynx; sarilumab available from Regeneron (the sequence is obtained from CN101454345B). The amino acid sequences are as follows:

Tocilizumab (Toci)
Tocilizumab heavy chain:
(SEQ ID NO: 8)
EVQLQESGPGLVRPSQTLSLTCTVSGYSITSDHAWSWVRQPPGRGLEWIG
YISYSGITTYNPSLKSRVTMLRDTSKNQFSLRLSSVTAADTAVYYCARSL
ARTTAMDYWGQGSLVTSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTIPPVL
DSDGSFFLYSKLTVDKSRWQQGNVFSCSVMHEALHNHYTQKSLSLSPGK
Tocilizumab light chain:
(SEQ ID NO: 9)
DIOMTQSPSSLSASVGDRVTFICRASQDISSYLNWYQQKPGKAPKLLIYY
TSRLHSGVPSRFSGSGSGTDFTFTISSLQPEDIATYYCQQGNTLPYTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
20A11-Fc fusion protein (20A11, wherien the VHH
seqeunce is derived from vobarilizumab)
(SEQ ID NO: 10)
EVQLVESGGGLVQPGGSLRLSCAASGSVFKINVMAWYRQAPGKGRELVAG
IISGGSTSYADSVKGRFTISRDNAKNTLYLQMNSLRPEDTAVYYCAFITT
ESDYDLGRRYWGQGTLVTVSSEPKSSDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQ0VSLTCLVKGFYPSDIAVEWESNGQPENNYKT
TPPVLDSDGSFFLYSKLTVDKSRWQOGNVFSCSVMHEALHNEYTQKSLSL
SPGK
Sarilumab (Sari)
heavy chain
(SEQ ID NO: 11)
EVQLVESGGGLVQPGRSLRLSCAASRFTFDDYAMFEWVRQAPGKGLEWVS
GISWNSGRIGYADSVKGRFTISRDNAENSLFLQMNGLRAEDTALYYCAKG
RDSFDIWGQGTMVTVSSASTKGPSVFPLAPSSKSTSGGTAALGCLVKDYF
PEPVTVSWNSGALTSGVHTFPAVLOSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
light chain
(SEQ ID NO: 12)
DIQMTQSPSSVSASVGDRVTITCRASQGISSWLAWYQQKPGKAPKLLIYGA
SSLESGVPSRFSGSGSGTDFTLTISSLQPEDFASYYCQQANSFPYTFGQGT
KLEIKRTVAAPSVFIFPPSDEQLKSGTASVVCLLNNFYPREAKVQWKVDNA
LQSGNSQESVTEQDSKDSTYSLSSTLTSKADYEKHKVYACEVTHQGLSSPV
TKSFNRGEC

HEK293E cells (i.e. 293E cells) were transiently transfected with PEI for protein expression. Then the purification method was selected according to the tags or fusion proteins as follows:

1. Nickel Column Purification (Isolation of His-tagged proteins): Supernatants containing the expressed products were high-speed centrifuged to remove impurities.

The nickel column was equilibrated with PBS buffer and rinsed with 2-5 column volumes; the supernatant samples were applied to Ni Sepharose Excel Column (GE Healthcare, Cat #17-3712-01) at a certain flow rate. The column was rinsed with PBS buffer until the A280 reading dropped to the baseline, then the column was rinse with PBS+10 mM imidazole to remove the undesired non-specifically bound proteins and the effluents were collected. Finally, the target proteins were eluted with PBS solution containing 300 mM imidazole and elution peaks were collected.

2. Chromatographic Exclusion Purification: SEC column (superdex75) was pre-equilibrated with PBS. Samples were loaded (the loading volume should be no more than 3% of the column volume) and then eluted with PBS as mobile phase. Each elution peak was collected and the target protein-containing fractions were identified by SDS-PAGE.

3. Flag Affinity Chromatography (Isolation of flag-containing proteins): The flag affinity filler was equilibrated with 0.5 x PBS and washed with 2-5 column volumes. The samples to be purified were incubated with the filler for 2 hours at room temperature. The filler was washed with 5 column volumes of 0.5×PBS, and the target proteins were eluted with TBS buffer containing 100 μg/ml 3×Flag polypeptide, collected, and identified by SDS-PAGE.

4. Affinity Chromatography of chimeric antibody (Isolation of Fc-containing proteins): Supernatants containing the expressed products were high-speed centrifuged to remove impurities, and the supernatants containing the expressed recombinant antibodies were purified using Protein A column. The column was rinse with PBS until the A280 reading dropped to the baseline. The target proteins were eluted with 100 mM acetic acid, pH 3.0 and neutralized with 1 M Tris-HCl, pH 8.0. The eluted sample was appropriately concentrated and further purified by gel chromatography Superdex 200 (GE) pre-equilibrated with PBS. The peak of the depolymerized product was collected and aliquoted for use.

### Example 2: Llama Immunization, Phage Display Library Construction and Screening

IL-6R.Fc or IL-6R.His, used as an immunogen, was mixed with Freund's complete adjuvant or Freund's incomplete adjuvant or PBS, and then was injected into alpacas 6 times. Peripheral blood samples were taken for titer determination before and after of 4^{th}, 5th and 6^{th} immunizations. Splenocytes were extracted after the last immunization.

The isolated lymphocytes were subjected to RNA extraction followed by reverse transcription into cDNAs. Two-step PCR was used for library construction: in first step, three forward primers designed on FR1 region and three reverse primers designed on CH2 region were mixed at equal ratio for PCR reaction. Small fragments were recovered from agarose gel. In the second step, both the forward and reverse primers were incorporated with sfiI restriction site and protective base. Except for the sfiI restriction site and the protective base, the sequences of forward primers were identical to the forward primers used in the first round PCR. The reverse primers were designed on FR4 region. The second step PCR products were recovered and cloned into phagemid vector, then electro-transformed into SS320 competent cells. Finally, a phage display library with capacity of 1.74E9 was obtained.

The constructed library was packaged with a helper phage to form a phage. The phage dsplay library was panned and screened for IL-6R binding. Desired clones were identified by ELISA. The panning was performed by both liquid phase and solid phase method: in the liquid phase method, biotinylated IL-6R.BP15 antigen bind to phage particles in the liquid phase and then were captured by streptavidin magnetic beads; in the solid phase method, ELISA plate was coated with IL-6R.Fcwhich was bound to phage particles. After two rounds of panning, monoclonal packed phages were picked up. The binding activity and the IL-6- and gp130-blocking activity, were determined by ELISA. The ELISA process is as follows:

IL-6R-binding ELISA: The plate was coated with 2 ng/μl streptavidin, 100 μl/well, overnight at 4° C., and blocked with 2% MPBSCaMg at 37° C. for 1 hour. Then the plate was washed and incubated with 1 ng/μl of IL-6R.BP15 at 37° C. for 1 hour. The plate was then washed followed by addition of 50 μl/well of 2% MPBSCaMg and 50 μl/well of phage supernatant. After Incubation at 37° C. for 1 hour, the plate was washed followed by addition of 100 μl of anti-M13 HRP with1:5000 dilution. After 1 hour incubationat 37° C. and wash, t100 μl/well of TMB was added for plate development. The reaction was stopped by addition of 100 μl of 1 M H₂SO₄. And signal were determined by at OD₄₅₀.

IL-6-blocking ELISA: The plate was coated with 100 ul/well of IL-6.Fc at 2 ng/μl, After overnight incubation at 4° C., the plate was blocked with 2% MPBSCaMg (2% milk, 0.90 mM CaCl₂, 0.49 mM MgCl₂, 1×PBS) at 37° C. for 1 hour. After washing, plate was incubated with 100 μl of IL-6R.his (1 ng/μl) at 37° C. for 1 hour. After that, the plate was washed and incubated with 50 μl/well of phage supernatant+50 μl/well of 2% MPBSCaMg blocking solutionat 37° C. for 1 hour. The plate was then washed followed by incubationwith 100 μl of anti-M13 HRP (GE healthcare, Cat# 27-9421-01), diluted at 1:5000, at 37° C. for 1 hour. After washing and TMB incubation,.the reaction was stopped by addition of 100 μl of 1 M H₂SO₄. And signal was read at OD₄₅₀.

Gp130-blocking ELISA: The plate was coated with 2 ng/μl gp130, 100 μl/well, overnight at 4° C., and blocked with 2% MPBSCaMg at 37° C. for 1 hour. Then the plate was washed and incubated with 100 μl 1 ng/μl IL-6R.his+1 ng/μl of IL-6R.his at 37° C. for 1 hour. After washing, 50 μl/well of phage supernatant+50 μl/well of 2% MPBSCaMg blocking solution were added followed by lhour incubattion at 37° C. for 1 hour. Then the plate was washed and 100 μl of anti-M13 HRP, diluted at 1:5000, was added in each well followed byincubation at 37° C. for 1 hour. After washing, the plate was developed with 100 μl/well of TMB. The reaction was stopped by addition of 100 μl of 1 M H₂SO₄. And signal was measured at OD₄₅₀.

Clones thata meet preselected criteria were selected for sequencing, such as: positive IL-6R-binding result, the reading of OD₄₅₀ (IL-6R-binding ELISA)/OD₄₅₀ (IL-6-blocking ELISA) (IL-6R/IL-6) was equal or higher than4; the reading of OD₄₅₀ (IL-6-blocking ELISA)/OD₄₅₀ (IL-6-blocking ELISA) (IL-6/gp130) was equal or greater than 3.

The sequences were analyzed and a panel of clones with unique sequences were identified

### Example 3: Construction and Characterization of Fc Fusion Proteins

In or to further characterize the selected clones at molecular and cellular level, the identified VHHs were fused to N-teminus of Fc, and cloned into mammalian expression vectors followed by transiently expressed in 293E cells. After protein A purification, VHH-hFc fusion proteins were subjected to the following tests.

IL-6R-blocking assay (Test Example 1), gp130-binding blocking assay (Test Example 2), U266B1 cell binding activity (Test Example 3), and IL-6-stimulated TF-1 proliferation blocking assay (Test Example 4). An IL-6R single domain antibody, 1764, which has the best properties in all aspects was selected. Its nucleotide sequence is shown as below:

(SEQ ID NO: 13)
gaggtgcagctggtggagtctgggggagggttggtgcaggctggggggtc
tctgagactctcctgtgaagcctctggaaacatcttcaagatcaatgtca
tgggctggtaccgccaggctccagggaagcagcgcgagtgggtcgcagct
attattagtggcggtagcacaaactalgcagactccgtgaagggccgatt
caccatctccagagacaacgccaagaacacggtgtatctgcaaatgaaca
gcctgaaacctgaggacacggccgtctattactgtaatgctattctcacc
tataacgaccatgacctagggtctgactactggggccagggcacccaggt
caccgtctcctca;

The encoded amino acid sequence is shown as below:

(SEQ ID NO: 14)
EVQLVESGGGLVQAGGSLRLSCEASGNIFKINVMGWYRQAPGKQREWVAA
IISGGSTNYADSVKGRFTISRDNAKNTVYLQMNSLKPEDTAVYYCNAILT
YNDYDLGSDYWGQGTQVTVSS;

wherein, the CDRs1-3 of the single domain antibody 1764 were shown in underlined text, and the sequences are:

CDR1:
(SEQ ID NO: 15)
INVMG;
CDR2:
(SEQ ID NO: 16)
AIISGGSTNYADSVKG;
CDR3:
(SEQ ID NO: 17)
ILTYNDYDLGSDY;

The above single domain antibody VHH was fused to the following Fc fragment to form a fusion protein:

Nucleotide sequence encoding the human Fc fragment is shown as follow:

(SEQ ID NO: 18)
gagcccaaatctagtgacaaaactcacacgtgcccaccgtgcccagcacc
tgaactcctggggggaccgtcagtcttcctcttccccccaaaacccaagg
acaccctcatgatcccccggacccctgaggtcacatgcgtggtggtggac
gtgagccacgaagaccctgaggcaagttcaactggtacgtggacggcgtg
gaggtgcataatgccaagacaaagccgcgggaagagcagtacaacagcac
gtaccgtgtggtcagcgtcctcaccgtcctgcaccaggactggctgaatg
gcaaggagtacaagtgcaaggtctccaacaaagccctcccagcccccatc
gagaaaaccatctccaaaagccaaagggcagccccgagaaccacaggtgt
acaccctgcccccatcccgggatgagctgaccaagaaccaggtcagcctg
acctgcctggtcaaaggcttctatcccagcgacatcgccgtggagtggga
gagcaatgggcagccggagaacaactacaagaccacgcctcccgtgctgg
actccgacggctccttcttcctctacagcaagctcaccgtggacaagagc
aggtggcagcaggggaacgtcttctcatgctccgtgatgcatgaggctct
gcacaaccactacacgcagaagagcctctccctgtctccgggtaaatga;

The human Fc fragment:

(SEQ ID NO: 19)
EPKSSDKTHTCPPCPAPELLGGPSVFLFPPKPKDTLMISRTPEVTCVVVD
VSHEDPEVKFNWYVVDGVEVHNAKTKPREEQYNSTYRVVSVLTVLHQDWL
NGKEYKCKVSNKALPAPIEKTISKAKGQPREPQVYTLPPSRDELTKNQVS
LTCLVKGFYPSDIAVEWESNGQPENNYKTTPPVLDSDGSFFLYSKUVDKS
RWQQGNVFSCSVMHEALHNHYTQKSLSLSPGK

### Example 4: Humanization of 1764

In order to avoid the anti-drug response of the therapeutic antibody, the Llama-derived molecule 1764 was humanized in the present disclosure to reduce its immunogenicity. Humanization was based on the sequences of 1764 and performed in a manner of gradual mutation toward human sequence, and the Liama-derived amino acid residues located on FR region of 1764 were gradually replaced with human-derived residues. The designed sequences are as follows.

TABLE 1
The designed sequences

| Variant | |
|---|---|
| No. | Mutation sites in 1764 variants |
| 1 | A14P, K86R |
| 2 | E23A, Q44G |
| 3 | A14P, K86R, Q116L |
| 4 | E23A, Q44G, Q116L |
| 5 | E23A, N96A, A97F, Q116L |
| 6 | A14P, E23A, Q44G, V78L, K86R, Q116L |
| 7 | A14P, E23A, V78L, K86R, N96A, A97F, Q116L |
| 8 | E23A, Q44G, V78L, N96A, A97F, Q116L |
| 9 | A14P, E23A, Q44G, V78L, K86R, N96A, A 97F, Q116L |

Note: For example, A14P indicates that the amino acid residue A at position 14 of 1764 was substituted by P. The numbering of the amino acid residues in this table is according to the natural position numbering as shown in SEQ ID NO: 14.

>1764-mu1
(SEQ ID NO: 20)
EVQLVESGGGLVQpGGSLRLSCEASGNIFKINVMGWYRQAPGKQREWVA
AIISGGSTNYADSVKGRFTISRDNAKNTVYLQMNSLrPEDTAVYYCNAI
LTYNDYDLGSDYWGQGTQVTVSS
>1764-mu2
(SEQ ID NO: 21)
EVQLVESGGGLVQAGGSLRLSCaASGNIFKINVMGWYRQAPGKgREWVA
AIISGGSTNYADSVKGRFTISRDNAKNTVYLQMNSLKPEDTAVYYCNAI
LTYNDYDLGSDYWGQGTQVTVSS
>1764-mu3
(SEQ ID NO: 22)
EVQLVESGGGLVQpGGSLRLSCEASGNIFKINVMGWYRQAPGKQREWVA
AIISGGSTNYADSVKGRRTISRDNAKNTVYLVANSLrPEDTAVYYCNAI
LTYNDYDLGSDYWGQGTLVTVSS
>1764-mu4
(SEQ ID NO: 23)
EVQLVESGGGLVQAGGSLRLSCAASGNIFKINVMGWYRQAPGKGREWVA
AIISGGSTNYADSVKGRFTISRDNAKNTVYLQMNSLKPEDTAVYYCNAI
LTYNDYDLGSDYWGQGTLNTVSS
>1764-mu5
(SEQ ID NO: 24)
EVQLVESGGGLVQAGGSLRLSCAASGNIFKINVMGWYRQAPGKQREWVA
AIISGGSTNYADSVKGRFTISRDNAKNTVTYLQMNSLKPEDTAVYYCAF
ILTYNDYDLGSDYWGQGTLVTVSS
>1764-mu6
(SEQ ID NO: 25)
EVQLVESGGGLVQPGGSLRLSCAASGNIFKINVMGWYRQAPGKGREWVA
AIISGGSTNYADSVKGRETBRDNAKNILYLQMNSLRPEDTAVYYCNAIL
TYNDYDLGSDYWGQTLVTVSS
>1764-mu7
(SEQ ID NO: 26)
EVQLVESGGGLVQPGGSLRLSCAASGNIFKINVMGWYRQAPGKQREWVA
AIISGGSTNYADSVKGRFTISRDNAKNTLYLQMNSLRPEDTAVYYCAFI
LTYNDYDLGSDYWGQGTLVTVSS
>1764-mu8
(SEQ ID NO: 27)
EVQLVESGGGLYQAGGSLRLSCAASGNIFKINVMGWYRQAPGKGREWVA
AIISGGSTNYADSVKGRFTISRDNAKNTLYLQMNSLKPEDTAVYYCAFI
LTYNDYDLGSDYWGQGTLVTVSS
>1764-mu9
(SEQ ID NO: 28)
EVQLVESGGGLVQpGGSLRLSCaASGNIFKINVMGWYRQAPKgREWVAA
IISGGSTNYADSVKGRFFISRDNAKNTIYLQMNSLrPEDTAVYYCafIL
TYNDYDLGSDYWGQGTIVTVSSEPKSS

The designed nine clones were fused to Fc (amino acid sequence was shown in SEQ ID NO: 19), constructed into mammalian transient expression vectors, and transiently expressed in 293E cells. After affinity purification, VHH-hFc fusion protein was obtained.

The purified IL-6R antibody fusion proteins (containing Fc fragment, hereinafter also referred to as IL-6R antibodies) were subjected to the following tests.

### Test Example 1: IL-6R Blocking Assay with IL-6R Antibodies

The formation of IL-6R/IL-6 complex can be blocked by the binding of IL-6R antibody to IL-6R, indicating the downstream cell signaling could be blocked by preventing IL-6/IL-6Rcomplex formationon the cell membrane. All of the expressed the purified antibodies were tested for their abilities to block the binding between IL-6 and IL-6R by ELISA. The procedures was descripted as follows:

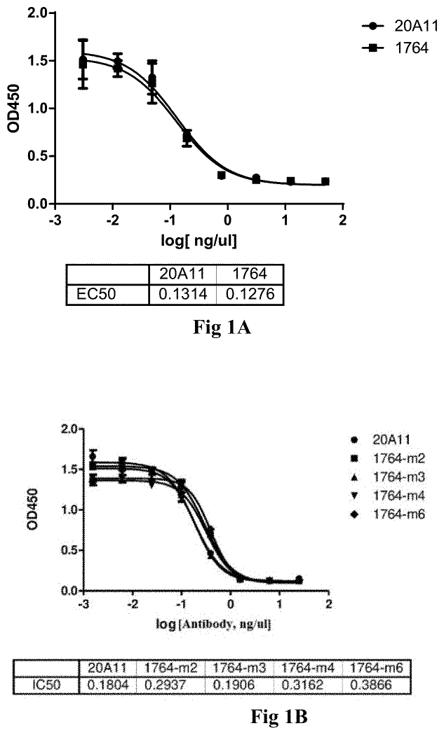

The plates were coated with100 μl of 2 ng/μl IL-6.Fc at 4° C. overnight, and blocked with 2% skim milk at 37° C. for 1 hour. The plates were then washed, incubated with 100 μl of serial diluted testing samples with and 1 ng/μl IL-6R.BP15. After 1 hourincubation at 37° C,. the plates were washed andHRP-streptavidin (Jackson, 016-030-084) diluted at 1:4000 was added to the plate followed by incubation. After wash and 100 μl TMB develoment at room temperature for 5 min. 100 μl of 1M H₂SO₄ was added to terminate the reaction. The OD₄₅₀ values were read. The results are shown in FIGS. 1A to 1C).

### Test Example 2: gp130-Binding Blocking Assay with IL-6R Antibodies

The complex of IL-6 and IL-6R can bind to GP130. The assay will evaluate if the selected antibodies were able to block the formation of IL-6/IL-6R/gp130 complex by binding to IL-6R. The plates were coated with 1 μg/ml gp130 (diluted in PBS) at 4° C. overnight. The coating solution was discarded, and plates were blocked with 5% skim milk in PBS at 37° C. for 2-3h. After blocking, the plates were washed 3 times with washing buffer PBST (0.1% Tween-20 in PBS).

IL-6.his and IL-6R.BP15 were diluted with 1% BSA in PBS to a concentration of 0.06 μg/ml. Starting at 50 ug/ml, IL-6R antibodies were 1:3 serially diluted in dilution buffer and Both testing antibody samples and IL-6/IL-6R complex were added to the plate followed by lh incubation at 37° C. After that, the plates were washed for 3 times with washing buffer PBST (0.1% Tween-20 in PBS).

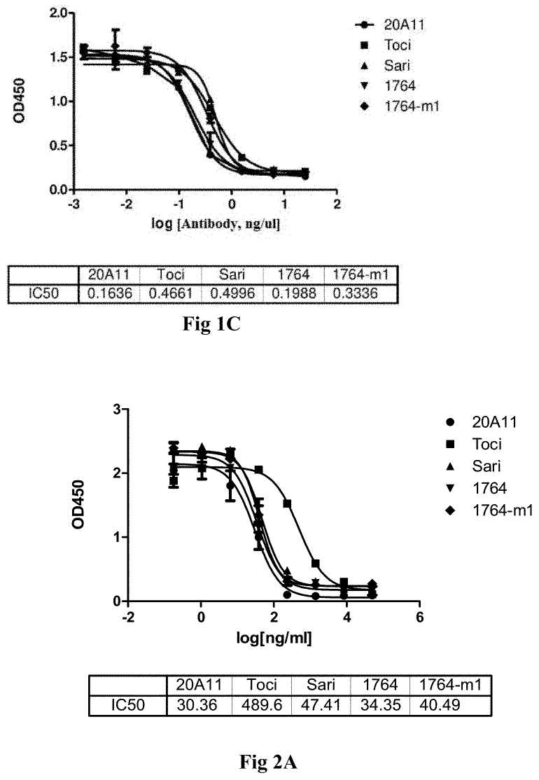

1:2000 diluted Secondary antibody HRP streptavidin (Jackson, 016-030-084) was added to the plate followed by 1 h incubation at 37° C.After incubation and wash,. TMB was used for development. OD₄₅₀ values were read. The results are shown in FIGS. 2A and 2B.

### Test Example 3: Binding Activities of IL-6R Antibodies to U266B1 Cells

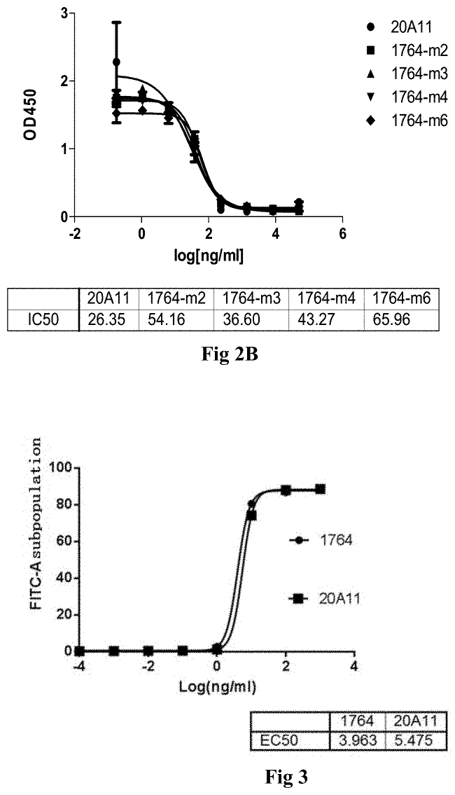

To test binding abilities of IL-6R antibodies to cell surface IL-6R, binding assay was performed on IL-6R-expressing U266B1 cells with IL-6R antibodies. U266B1 cells (ATCC® TIB-196™) were collected, centrifuged at 400g, 4° C. for 5min. Pre-cooled DPBS containing final concentration of 10% FBS was used to resuspend cell pellet followed by centrifuged at 400g, 4° C. for 5min,.After two times of wash, twice. cells were seeded onto 96-well plates, 10E⁵/well. Each well was loaded with 50 μl of sample, and incubated at 4° C. for 45-60 minutes followed by additionof 250 μl pre-cooled DPBS containing final concentration of 10% FBS. After wash for two times 50 μl of secondary antibody Alexa Fluor@488 goat anti-human IgG (H+L) (Lifetechologies, A11013) (diluted at 1:200) was added and incubated at 4° C. in the darkness for 45 minutes. After that, the plates were washedwith 250 μl/well of pre-cooled DPBS containing final concentration of 10% FBS for two times. Each well.100 μl/well of pre-cooled DPBS were added for cell resuspension. Detection was performed on machine(BD, FACSverse). Results were analyzed with flowjo and were shown in FIG. 3.

### Test Example 4: Inhibition of IL-6-Stimulated TF-1 Proliferation by IL-6R Antibodies

Through binding to membrane-type IL-6R, IL-6 can initiate downstream signaling and induce proliferation of TF-1 cells. IL-6R antibodies can block the binding of IL-6 to IL-6R, thereby inhibit the proliferation of TF-1 cells.

TF-1 cell culture: TF-1 leukemia cells (ATCC, CRL-2003) were cultured in RPMI1640 containing 10% FBS (2ng/mL rhGM-CSF) (GE, Catalog No. SH30809.01), and placed in incubator at 37° C., 5% CO₂, at the cell density not above 1E6 cells/ml.

The procedures of assay for inhibiting IL-6-stimulated TF-1 proliferation: Cells in logarithmic growth phase were washed three times with PBS, centrifuged at 800 rpm for 3 min, and the cell density was adjusted with RPMI1640 (FBS 2%, recombinant human IL-6, 5 ng/ml) to 20,000 cells/well/180 μl. 20 μl of serial dilutions of the test antibodies were added to 96-well plates and incubated for 3 days (the starting antibody concentration of the serial dilution was 490 nM, and 10-fold serially diluted to 4.9×10⁻⁷ nM). 100 μl of cell suspension was mixed with 100 μl of Cell Titer (Promega, Catalog No. G7573) for detection. The results are shown in Table 2.

TABLE 2
The blocking effect of IL-6R antibodies on IL-6-stimulated
TF-1 proliferation

| | | | 1764- | 1764- | 1764- | 1764- | 1764- |
|---|---|---|---|---|---|---|---|
| EC50 | 20A11 | 1764 | mu1 | mu2 | mu3 | mu4 | mu6 |
| (nM) | 0.3545 | 0.4100 | 0.4469 | 0.3442 | 0.3365 | 0.3704 | 0.6032 |

### Test Example 5: IL-6R Antibodies Inhibit IL-6-Induced Hepcidin Expression

High expression of hepcidin is major cause of inflammatory anemia. IL-6 will up-regulate hepcidin expression by addition ofo IL-6R to activate the downstream signaling pathways. Therefore, IL-6R antibodies may inhibit IL-6-induced hepcidin expression by blocking the binding between IL-6 and IL-6R. The experimental design is as follows:

Group 1: blank control;

Group 2: positive control, treat with IL-6 only;

Group 3: treat with 1764-mu3 and IL-6;

Group 4: treat with Tocilizumab and IL-6;

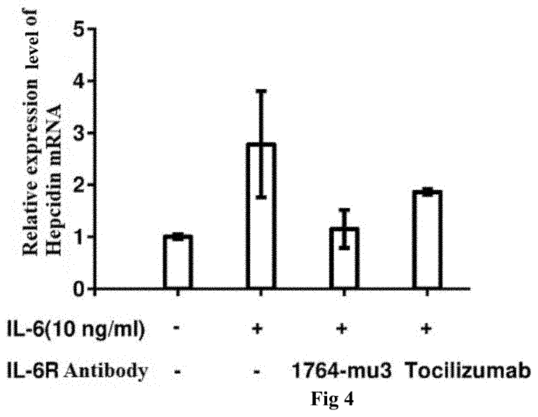

Hep3B cells (Chinese Academy of Sciences, TCHu106) were cultured in EME medium (Gibco, 11095098) containing 10% FBS, and incubated in an incubator at 37° C., 5% CO₂. Cell passage was performed every 2 to 3 days. Hep3B cells in logarithmic growth phase were plated in 6-well plates at 200,000 cells per well and cultured in an incubator at 37° C., 5% CO₂. On the next day, when cell confluency reached 80% to 90%, IL-6R antibody 1764-mu3 and Tocilizumab were added to each well in Group 3 and Group 4, respectively, at a final concentration of 5 nM. After incubation in an incubator at 37° C., 5% CO₂ for 30 min, each well was added with IL-6 protein at a final concentration of 10 ng/ml, except for the blank control. The cells were further cultivated for 24 hours, and then the cells were collected, total RNA was extracted, and reverse transcribed into cDNA. Fluorescent quantitative PCR was performed by using cDNA as a template to detect the mRNA level of hepcidin, with the expression level of microglobulin mRNA as an internal reference. The results are shown in Table 3 and FIG. 4.

TABLE 3
The inhibition of IL-6R antibodies on IL-6-induced hepcidin
expression level

| Sample name | Group 1 | Group 2 | Group 3 | Group 4 |
|---|---|---|---|---|
| Relative | 1 | 2.783 | 1.154 | 1.866 |
| expression level of | | | | |
| hepcidin mRNA | | | | |
| (fold) | | | | |

### Test Example 6: BIAcore Affinity (ED) Assay of the Antibodies

Human anti-capture antibodies were covalently coupled onto Biosensor chip CM5 (Cat.# BR-1000-12, GE) in a Biacore instrument (Biacore T200, GE) with Human Anti-Capture Kit (Cat. #BR-1008-39, GE) according to manufacturer's protocol, thereby certain amounts of antibodies to be tested were captured. Antigens (hIL-6R.his and cIL-6R.FH) at a series of concentration gradients were then flowed through the surface of the chip . The reaction signals were detected in real time using Biacore instrument (Biacore T200, GE) to obtain binding and dissociation curves.

After each cycle of dissociation, the biochips were washed and regenerated with the regeneration solution provided in the Human Anti-Capture Kit. The amino coupling kit used in the experiment was purchased from GE (Cat. #BR-1000-50, GE) and the buffer was HBS-EP+10×buffer solution (Cat.#BR-1006-69, GE), diluted to 1×(pH 7.4) with D.I. water.

The experimental data were fitted with BlAevaluation version 4.1 and GE software (1:1) Langmuir model to obtain the affinity values. The results are shown in Table 4.

TABLE 4
The result showing the affinity of 1764 and each humanized clone for
hIL-6R detected in BIAcore Assay

| | hIL-6R | | |
|---|---|---|---|
| sample | ka (1/Ms) | kd (1/s) | KD (M) |
| 1764 | 1.28E+06 | 1.64E−04 | 1.29E−10 |
| 1764-mu1 | 1.23E+06 | 1.28E−04 | 1.05E−10 |
| 1764-mu2 | 1.23E+06 | 1.54E−04 | 1.26E−10 |
| 1764-mu3 | 1.21E+06 | 1.54E−04 | 1.27E−10 |
| 1764-mu4 | 1.20E+06 | 1.50E−04 | 1.25E−10 |
| 1764-mu5 | 1.10E+06 | 1.11E−04 | 1.01E−10 |
| 1764-mu6 | 1.23E+06 | 2.45E−04 | 2.00E−10 |
| 1764-mu7 | 8.97E+05 | 1.17E−04 | 1.31E−10 |
| 1764-mu8 | 1.01E+06 | 1.44E−04 | 1.43E−10 |
| 1764-mu9 | 8.53E+05 | 1.21E−04 | 1.42E−10 |

### Test Example 7. Pharmacokinetic Experiment in Rats

Pharmacokinetic experiment of antibody fusion proteins were performed in rats. The dosing schedule to rats were as follows:

TABLE 5
Dosing schedule

| Animal Group Number | B | C |
|---|---|---|
| Compound Name | 1764-mu3 | 1764-mu4 |
| Dosing route | i.v. | i.v. |
| formulation | PBS, pH 7.4, saline | |
| Adminstration Dosage | 5 | 5 |
| (mg/kg) | | |
| Adminstration Volume | 5 | 5 |
| (ml/kg) | | |
| Drug Concentration | 1 | 1 |
| (mg/ml) | | |

12 SD rats (Sipper-Beikai Experimental Animal Co., Ltd.) (equal number of male and female) were divided into 2 groups, 6 rats in each group. The drugs were intravenously injected. In treatment group, 0.2 ml of whole blood samples were collected without addition of anticoagulant before administration as well as 15 min, 8 h, 1 day, 2 days, 4 days, 7 days, 10 days, 14 days, 21 days and 28 days of post administration. Blood samples were placed at 4° C. for 30 min, centrifuged at 1000 g for 15 min, and the supernatants (serum) were placed in EP tubes and stored at −80° C. The concentration of the target was determined by ELISA, and the pharmacokinetic parameters of the test drugs were calculated using Winnolin software. The test results are shown in Table 6.

TABLE 6
Pharmacokinetic parameters

| | 1764-mu3 | 1764-mu4 |
|---|---|---|
| dosage | 5 mg/kg | 5 mg/kg |
| Tₘₐₓ (h) | 0.25 | 0.25 |
| t_{1/2} (h) | 185 ± 17 | 148 ± 9 |

1764-mu3 and 1764-mu4 are single-chain antibodies with half-lives of 7.7 days and 6.2 days, respectively.

### Test Example 8. Test for Accelerated Stability

In order to observe the stability of the antibodies, an accelerated test was performed on some antibodies. 1 mg/ml antibody fusion protein was placed at 40° C. for 3 days, and then was observed for the presence or absence of precipitation, and were subjected to SEC detection. The test results are shown in Table 7.

TABLE 7
Accelerated experimental results

| | | 40° C. for |
|---|---|---|
| clone | SEC purity (%) | 3 days |
| 1764 | 96.97 | clear |
| 1764-mu1 | 98.09 | clear |
| 1764-mu2 | 95.84 | clear |
| 1764-mu3 | 98.21 | clear |
| 1764-mu4 | 94.98 | clear |

For clear understanding, the above invention has been described in detail with the aid of the accompanying drawings and examples. However, the description and examples should not be construed as limiting the scope of the disclosure. The numerical units in all figures are the same as the units indicated on the abscissa. All patents and scientific literatures cited herein are expressly incorporated by reference in their entirety.

1-20. (canceled)

21. An anti- human IL-6R monoclonal antibody or antigen-binding fragment thereof comprising CDR1, CDR2 and CDR3, wherein
the sequence of CDR1 is a sequence as set forth in SEQ ID NO: 15 or a variant sequence having 3, 2 or 1 amino acid differences from the sequence as set forth in SEQ ID NO: 15;
the sequence of CDR2 is a sequence as set forth in SEQ ID NO: 16 or a variant sequence having 3, 2 or 1 amino acid differences from the sequence as set forth in SEQ ID NO: 16; and
the sequence of CDR3 is a sequence as set forth in SEQ ID NO: 17 or a variant sequence having 3, 2 or 1 amino acid differences from the sequence as set forth in SEQ ID NO: 17.

22. The anti-human IL-6R monoclonal antibody or antigen-binding fragment thereof according to claim 21, wherein
the CDR1 sequence is as set forth in SEQ ID NO: 15;
the CDR2 sequence is as set forth in SEQ ID NO: 16; and
the CDR3 sequence is as set forth in SEQ ID NO: 17.

23. The anti- human IL-6R monoclonal antibody or antigen-binding fragment thereof according to claim 21, wherein the monoclonal antibody is selected from the group consisting of an alpaca-derived antibody, a chimeric antibody, and a humanized antibody.

24. The anti- human IL-6R monoclonal antibody or antigen-binding fragment thereof according to claim 23, wherein the monoclonal antibody comprises a VHH as set forth in SEQ ID NO: 14 or the humanized antibody comprises a variant of the VHH as set forth in SEQ ID NO: 14.

25. The anti-human IL-6R monoclonal antibody or antigen-binding fragment thereof according to claim 24, wherein:
the variant of VHH has 1 to 15 humanized amino acid mutation(s) in a FR region of the VHH as set forth in SEQ ID NO: 14.

26. The anti- human IL-6R monoclonal antibody or antigen-binding fragment thereof according to claim 25, wherein: the amino acid mutations are selected from the group consisting of: A14P, E23A, Q44G, V78L, K86R, N96A, A97F, Q116L or combinations thereof

27. The anti- human IL-6R monoclonal antibody or antigen-binding fragment thereof according to claim 26, wherein the variant of VHH is selected from the group consisting of SEQ ID NO: 20, 21, 22, 23, 24, 25, 26, 27 and 28.

28. The anti- human IL-6R monoclonal antibody or antigen-binding fragment thereof according to claim 21, wherein the antibody further comprises a human antibody Fc region.

29. The anti- human IL-6R monoclonal antibody or antigen-binding fragment thereof according to claim 28, wherein the antibody comprises a human antibody Fc region as set forth in SEQ ID NO: 19.

30. The anti- human IL-6R monoclonal antibody or antigen-binding fragment thereof according to claim 21, wherein the antigen-binding fragment is a single domain antibody or a peptide comprising the three CDRs.

31. An isolated monoclonal antibody or antigen-binding fragment thereof, which competes with the monoclonal antibody or antigen-binding fragment thereof according to claim 21 for binding to IL-6R and blocks the binding of IL-6 to IL-6R.

32. A pharmaceutical composition comprising:
a therapeutically effective amount of the monoclonal antibody or antigen-binding fragment thereof according to claim 21, and
one or more pharmaceutically acceptable carriers, diluents or excipients.

33. A nucleic acid molecule encoding the monoclonal antibody or antigen-binding fragment thereof according to claim 21.

34. The nucleic acid molecule of claim 33 comprising:
a polynucleotide having at least 80% homology to the nucleic acid molecule as set forth in SEQ ID NO: 13.

35. A recombinant vector comprises the nucleic acid molecule of claim 33.

36. A host cell, transformed with the recombinant vector of claim 35, the host cell is a prokaryotic cell or a eukaryotic cell.

37. A method for producing an anti-human IL-6R monoclonal antibody or antigen-binding fragment thereof, the method comprising the steps of:
cultivating the host cell of claim 36;
forming and accumulating the monoclonal antibody or antigen-binding fragment thereof in culture; and
recovering the monoclonal antibody or antigen-binding fragment thereof from the culture.

38. A method for detecting or determining human IL-6R, comprising the step of:
contacting the monoclonal antibody or antigen-binding fragment thereof according to claim 21 with a sample.

39. An agent for detecting or determining human IL-6R, comprising:
the monoclonal antibody or antigen-binding fragment thereof according claim 21.

40. A method of treating or preventing a disease associated with human IL-6R in a subject in need thereof, the method comprising administering to the subject a monoclonal antibody or antigen-binding fragment thereof according to claim 21, wherein the disease associated with human IL-6R is selected from the group consisting of: sepsis, multiple myeloma, renal cell carcinoma, plasma cell leukemia, lymphoma, B-lymphoid hyperplasia, prostate cancer, osteoporosis, cachexia, psoriasis, glomerular mesangial proliferative glomerulonephritis, Kaposi's sarcoma, AIDS-related lymphoma, rheumatoid arthritis, systemic onset of juvenile idiopathic arthritis, hypergammaglobulinemia, regional enteritis, ulcerative colitis, systemic lupus erythematosus, multiple sclerosis, Castleman's disease, IgMγ-globulin disease, cardiac myxoma, asthma, autoimmune insulin-dependent diabetes and inflammatory anemia.


SEQUENCE LISTING (28 sequences)

SEQ ID NO: 1 - protein, 352 aa, artificial sequence
  hIL-6R.His
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPHHHHHH

SEQ ID NO: 2 - protein, 378 aa, artificial sequence
  hIL-6R.BP15
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPGSSDYKDDDDKHHHHHHGLNDIFEAQKIEWHE

SEQ ID NO: 3 - protein, 578 aa, artificial sequence
  hIL-6R.Fc
LAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 363 aa, artificial sequence
  cIL-6R.FH
LAPGGCPAQEVARGVLTSLPGDSVTLTCPGGEPEDNATVHWVLRKPAVGSHLSRWAGVGRRLLLRSVQLHDSGNYSCYRAGRPAGTVHLLVDVPPEEPQLSCFRKSPLSNVVCEWGPRSTPSPTTKAVLLVRKFQNSPAEDFQEPCQYSQESQKFSCQLAVPEGDSSFYIVSMCVASSVGSKLSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPTQAPTTNKDDDNILSRDSANATSLPVQDSSSVPLPGSSDYKDDDDKHHHHHH

SEQ ID NO: 5 - protein, 415 aa, artificial sequence
  IL-6.Fc
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQMEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 189 aa, artificial sequence
  IL-6.his
VPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQMHHHHHH

SEQ ID NO: 7 - protein, 829 aa, artificial sequence
  gp130.Fc
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 449 aa, artificial sequence
  Tocilizumab heavy chain
EVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 214 aa, artificial sequence [sequence omitted]

SEQ ID NO: 10 - protein, 353 aa, artificial sequence
  20A11-Fc fusion protein (20A11)
EVQLVESGGGLVQPGGSLRLSCAASGSVFKINVMAWYRQAPGKGRELVAGIISGGSTSYADSVKGRFTISRDNAKNTLYLQMNSLRPEDTAVYYCAFITTESDYDLGRRYWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 446 aa, artificial sequence
  Sarilumab (Sari) heavy chain
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, artificial sequence
  Sarilumab (Sari) light chain
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 363 bp, artificial sequence
  1764 VHH coding nucleic acid
gaggtgcagctggtggagtctgggggagggttggtgcaggctggggggtctctgagactctcctgtgaagcctctggaaacatcttcaagatcaatgtcatgggctggtaccgccaggctccagggaagcagcgcgagtgggtcgcagctattattagtggcggtagcacaaactatgcagactccgtgaagggccgattcaccatctccagagacaacgccaagaacacggtgtatctgcaaatgaacagcctgaaacctgaggacacggccgtctattactgtaatgctattctcacctataacgactatgacctagggtctgactactggggccaggggacccaggtcaccgtctcctca

SEQ ID NO: 14 - protein, 121 aa, artificial sequence
  1764 VHH
EVQLVESGGGLVQAGGSLRLSCEASGNIFKINVMGWYRQAPGKQREWVAAIISGGSTNYADSVKGRFTISRDNAKNTVYLQMNSLKPEDTAVYYCNAILTYNDYDLGSDYWGQGTQVTVSS

SEQ ID NO: 15 - protein, 5 aa, artificial sequence
  1764 CDR1
INVMG

SEQ ID NO: 16 - protein, 16 aa, artificial sequence
  1764 CDR2
AIISGGSTNYADSVKG

SEQ ID NO: 17 - protein, 13 aa, artificial sequence
  1764 CDR3
ILTYNDYDLGSDY

SEQ ID NO: 18 - DNA, 699 bp, artificial sequence
  human Fc fragment
gagcccaaatctagtgacaaaactcacacgtgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaagagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 19 - protein, 232 aa, artificial sequence
  human Fc fragment
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 121 aa, artificial sequence
  1764-mu1
EVQLVESGGGLVQPGGSLRLSCEASGNIFKINVMGWYRQAPGKQREWVAAIISGGSTNYADSVKGRFTISRDNAKNTVYLQMNSLRPEDTAVYYCNAILTYNDYDLGSDYWGQGTQVTVSS

SEQ ID NO: 21 - protein, 121 aa, artificial sequence
  1764-mu2
EVQLVESGGGLVQAGGSLRLSCAASGNIFKINVMGWYRQAPGKGREWVAAIISGGSTNYADSVKGRFTISRDNAKNTVYLQMNSLKPEDTAVYYCNAILTYNDYDLGSDYWGQGTQVTVSS

SEQ ID NO: 22 - protein, 121 aa, artificial sequence
  1764-mu3
EVQLVESGGGLVQPGGSLRLSCEASGNIFKINVMGWYRQAPGKQREWVAAIISGGSTNYADSVKGRFTISRDNAKNTVYLQMNSLRPEDTAVYYCNAILTYNDYDLGSDYWGQGTLVTVSS

SEQ ID NO: 23 - protein, 121 aa, artificial sequence
  1764-mu4
EVQLVESGGGLVQAGGSLRLSCAASGNIFKINVMGWYRQAPGKGREWVAAIISGGSTNYADSVKGRFTISRDNAKNTVYLQMNSLKPEDTAVYYCNAILTYNDYDLGSDYWGQGTLVTVSS

SEQ ID NO: 24 - protein, 121 aa, artificial sequence
  1764-mu5
EVQLVESGGGLVQAGGSLRLSCAASGNIFKINVMGWYRQAPGKQREWVAAIISGGSTNYADSVKGRFTISRDNAKNTVYLQMNSLKPEDTAVYYCAFILTYNDYDLGSDYWGQGTLVTVSS

SEQ ID NO: 25 - protein, 121 aa, artificial sequence
  1764-mu6
EVQLVESGGGLVQPGGSLRLSCAASGNIFKINVMGWYRQAPGKGREWVAAIISGGSTNYADSVKGRFTISRDNAKNTLYLQMNSLRPEDTAVYYCNAILTYNDYDLGSDYWGQGTLVTVSS

SEQ ID NO: 26 - protein, 121 aa, artificial sequence
  1764-mu7
EVQLVESGGGLVQPGGSLRLSCAASGNIFKINVMGWYRQAPGKQREWVAAIISGGSTNYADSVKGRFTISRDNAKNTLYLQMNSLRPEDTAVYYCAFILTYNDYDLGSDYWGQGTLVTVSS

SEQ ID NO: 27 - protein, 121 aa, artificial sequence
  1764-mu8
EVQLVESGGGLVQAGGSLRLSCAASGNIFKINVMGWYRQAPGKGREWVAAIISGGSTNYADSVKGRFTISRDNAKNTLYLQMNSLKPEDTAVYYCAFILTYNDYDLGSDYWGQGTLVTVSS

SEQ ID NO: 28 - protein, 126 aa, artificial sequence
  1764-mu9
EVQLVESGGGLVQPGGSLRLSCAASGNIFKINVMGWYRQAPGKGREWVAAIISGGSTNYADSVKGRFTISRDNAKNTLYLQMNSLRPEDTAVYYCAFILTYNDYDLGSDYWGQGTLVTVSSEPKSS